(19) United States
(12) Patent Application Publication
(10) Pub. No.: US 2022/0257195 A1
(43) Pub. Date: Aug. 18, 2022

(54) AUDIO PILL

(71) Applicant: Fraunhofer-Gesellschaft zur Förderung der angewandten Forschung e.V., Munich (DE)

(72) Inventor: Christoph SLADECZEK, Ilmenau (DE)

(21) Appl. No.: 17/661,974

(22) Filed: May 4, 2022

Related U.S. Application Data
(63) Continuation of application No. PCT/EP2020/081005, filed on Nov. 4, 2020.

(30) Foreign Application Priority Data
Nov. 6, 2019 (DE) 102019217145.1

(51) Int. Cl. A61B 5/00; A61B 7/02

(52) U.S. Cl. CPC A61B 5/6861; A61B 7/023; A61B 5/0086; A61B 5/002; A61B 2560/0219; A61B 2562/0219; A61B 2562/0204; A61B 2562/043; A61B 5/7264

(57) ABSTRACT

A sensor pill in accordance with an embodiment, suitable for being swallowed by a patient is provided. The sensor pill has a casing. Additionally, the sensor pill has one or more sensors. The one or more sensors have a microphone and/or an acceleration sensor.

### CROSS-REFERENCE TO RELATED APPLICATIONS

This application is a continuation of copending International Application No. PCT/EP2020/081005, filed Nov. 4, 2020, which is incorporated herein by reference in its entirety, and additionally claims priority from German Application No. 102019217145.1, filed Nov. 6, 2019, which is also incorporated herein by reference in its entirety.

### TECHNICAL FIELD

The application relates to an apparatus and to a method for supporting a medical examination and, in particular, to an audio pill.

### BACKGROUND OF THE INVENTION

In a general medical examination, diagnosis is frequently performed by means of “monitoring” by means of a stethoscope.

The state of organs and body parts (like heart, lungs, blood vessels, for example) can be identified directly using the noise caused.

A prerequisite for successfully determining the state of organs and body parts is the well-being (like concentration, no listening impairment, etc., for example), the experience and good training of the physician.

In particular the first factor cannot always be guaranteed in a medical practice, which is why a more objective evaluation is sought.

At present, there is no technical solution for this problem.

Instead, interpreting and evaluating the noises heard by the doctor is solely in the hands of the doctor.

WO 2012/006454A2 shows an apparatus capable of detecting one or more chemical parameters, the apparatus comprising a casing, a light source and an imaging device. The apparatus may be configured to be a capsule which can be swallowed.

U.S. Pat. No. 8,632,459 B2 and EP 1 967 125 A1 show a capsule endoscope which can be caused to float at an interface between a first liquid and a second liquid and ensures a sufficient field of view within the stomach.

US 2004/176685 A1 shows a capsule apparatus for medical purposes having a sensor.

JP 2010 035746 A shows a capsule endoscope comprising an acceleration sensor integrated into the capsule endoscope 10 to calculate the position of the capsule endoscope.

### SUMMARY

According to an embodiment, a sensor pill suitable for being swallowed by a patient may have: a casing of the sensor pill, and one or more sensors, wherein the one or more sensors have a microphone, wherein said microphone is a microphone of a plurality of microphones forming an array of microphones, the sensor pill having the array of microphones, wherein the array of microphones is configured to determine first directional information relating to a first of the acoustic noises detected by the array of microphones, and wherein the array of microphones is configured to determine second directional information relating to a second of the acoustic noises detected by the array of microphones.

According to another embodiment, a system may have: an inventive sensor pill as mentioned above, and an evaluation unit, the evaluation unit being configured to evaluate data detected by at least one of the one or more sensors of the inventive sensor pill as mentioned above.

According to another embodiment, a method may have the steps of: swallowing a sensor pill by a patient, the sensor pill having a casing and one or more sensors which have a microphone, and detecting an acoustic noise and/or a vibration by at least one of the one or more sensors of the sensor pill, wherein said microphone is a microphone of a plurality of microphones forming an array of microphones, the sensor pill having the array of microphones, wherein the array of microphones is configured to determine first directional information relating to a first of the acoustic noises detected by the array of microphones, and wherein the array of microphones is configured to determine second directional information relating to a second of the acoustic noises detected by the array of microphones.

Another embodiment may have a non-transitory digital storage medium having stored thereon a computer program for performing a method having the steps of: swallowing a sensor pill by a patient, the sensor pill having a casing and one or more sensors which have a microphone, and detecting an acoustic noise and/or a vibration by at least one of the one or more sensors of the sensor pill, wherein said microphone is a microphone of a plurality of microphones forming an array of microphones, the sensor pill having the array of microphones, wherein the array of microphones is configured to determine first directional information relating to a first of the acoustic noises detected by the array of microphones, and wherein the array of microphones is configured to determine second directional information relating to a second of the acoustic noises detected by the array of microphones, when said computer program is run by a computer.

A sensor pill in accordance with an embodiment, suitable for being swallowed by a patient is provided. The sensor pill comprises a casing. Additionally, the sensor pill comprises one or more sensors. The one or more sensors comprise a microphone and/or an acceleration sensor.

Additionally, an evaluation unit in accordance with an embodiment is provided. The evaluation unit is configured to evaluate data detected by at least one of the one or more sensors of the sensor pill described above.

Furthermore, a system in accordance with an embodiment is provided. The system comprises the sensor pill in accordance with an embodiment described above. Additionally, the system comprises the evaluation unit in accordance with an embodiment described above. The evaluation unit is configured to evaluate data detected by the one or more sensors of the sensor pill.

Additionally, a method in accordance with an embodiment is provided. The method comprises:
Swallowing a sensor pill by a patient, wherein the sensor pill comprises a casing and one or more sensors having a microphone and/or an acceleration sensor. And:
Detecting an acoustic noise and/or a vibration by at least one of the one or more sensors of the sensor pill.

Furthermore, a method in accordance with an embodiment is provided. The method comprises:
Evaluating data detected by at least one of the one or more sensors of the sensor pill described above.

Additionally, a method in accordance with an embodiment is provided. The method comprises:
Swallowing a sensor pill by a patient, wherein the sensor pill comprises a casing and one or more sensors having a microphone and/or an acceleration sensor.
Detecting an acoustic noise and/or a vibration by at least one of the one or more sensors of the sensor pill. And:
Evaluating the detected acoustic noises and/or the vibrations by an evaluation unit.

Additionally, a computer program having a program code for performing one of the methods described above is provided.

### BRIEF DESCRIPTION OF THE DRAWINGS

Embodiments of the invention will be described below referring to the drawing, in which:

FIG. 1 shows a sensor pill in accordance with an embodiment, suitable for being swallowed by a patient;

FIG. 2 shows a sensor pill in accordance with an embodiment, comprising a microphone;

FIG. 3 shows a sensor pill in accordance with an embodiment, comprising an acceleration sensor;

FIG. 4 shows a sensor pill in accordance with an embodiment, comprising an array of microphones as one of the one or more sensors;

FIG. 5 shows such a sensor pill in accordance with an embodiment comprising two sensors;

FIG. 6 shows a sensor pill in accordance with an embodiment, comprising an optical sensor as a second sensor;

FIG. 7 shows a sensor pill in accordance with an embodiment, comprising an optical sensor as a second sensor, additionally having a light source;

FIG. 8 shows a sensor pill in accordance with an embodiment in which the optical sensor is an infrared sensor 132;

FIG. 9 shows a sensor pill in accordance with an embodiment in which the optical sensor is an ultrasonic sensor 133;

FIG. 10 shows a sensor pill comprising a transmitter 151 in accordance with an embodiment;

FIG. 11 exemplarily shows how a receiver localizes the position of the sensor pill in accordance with an embodiment by means of near-field communication;

FIG. 12 shows a sensor pill 100 comprising a current source in accordance with an embodiment;

FIG. 13 shows an evaluation unit in accordance with an embodiment;

FIG. 14 shows an evaluation unit in accordance with an embodiment, additionally comprising a receiver for receiving data from a transmitter of the sensor pill; and

FIG. 15 shows a system in accordance with an embodiment.

### DETAILED DESCRIPTION OF THE INVENTION

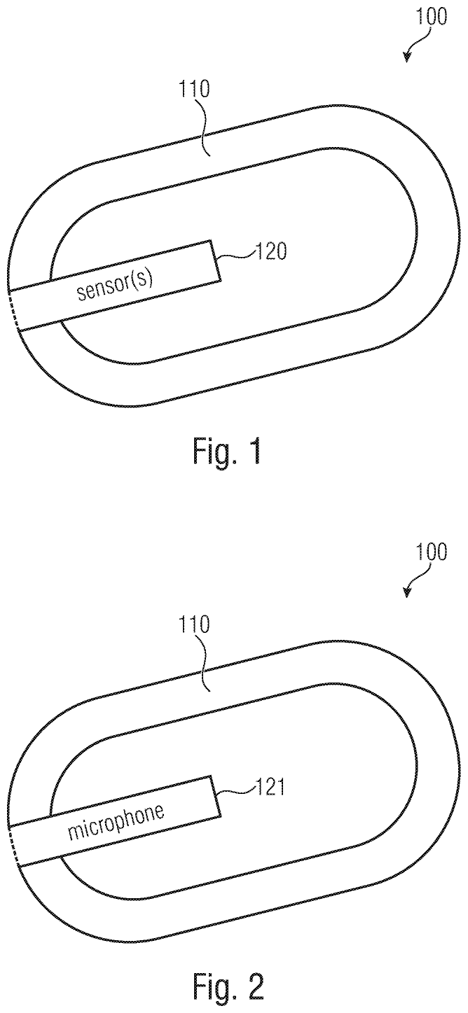

FIG. 1 shows a sensor pill 100 in accordance with an embodiment, suitable for being swallowed by a patient.

The sensor pill 100 comprises a casing 110.

Furthermore, the sensor pill 100 comprises one or more sensors 120. The one or more sensors 120 comprise a microphone 121 and/or an acceleration sensor 125.

FIG. 2 shows a sensor pill 100 in accordance with an embodiment, comprising a microphone 121 as one of the one or more sensors.

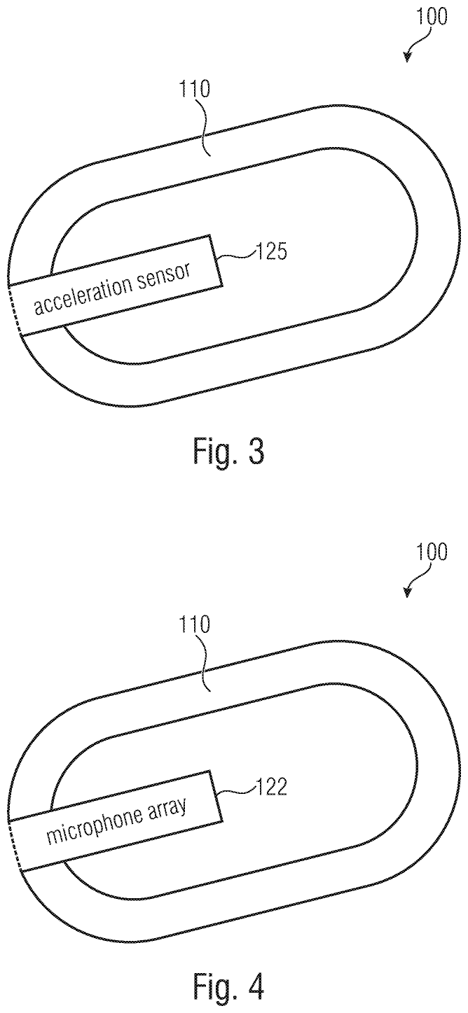

FIG. 3 shows a sensor pill 100 in accordance with an embodiment, comprising an acceleration sensor 125 as one of the one or more sensors.

In accordance with an embodiment, the microphone 121 may, for example, be configured to record acoustic noises originating from inside the body of the patient.

In one embodiment, said microphone 121 may, for example, be a microphone of a plurality of microphones which form an array 122 of microphones. The sensor pill 100 may, for example, comprise the array of microphones.

FIG. 4 shows a sensor pill 100 in accordance with an embodiment, comprising an array 122 of microphones as one of the one or more sensors. The microphone 121 referred to in FIG. 2 is one of the microphones of the array 122 of microphones.

In accordance with an embodiment, the array 122 of microphones may, for example, be configured to determine first directional information relating to a first of the acoustic noises detected by the array 122 of microphones. Thus, the array 122 of microphones may, for example, be configured to determine second directional information relating to a second of the acoustic noises detected by the array 122 of microphones.

In one embodiment, the acceleration sensor 125 may, for example, be configured to detect vibrations which originate from inside the body of the patient.

In accordance with an embodiment, the one or more sensors 120 may, for example, be two or more sensors.

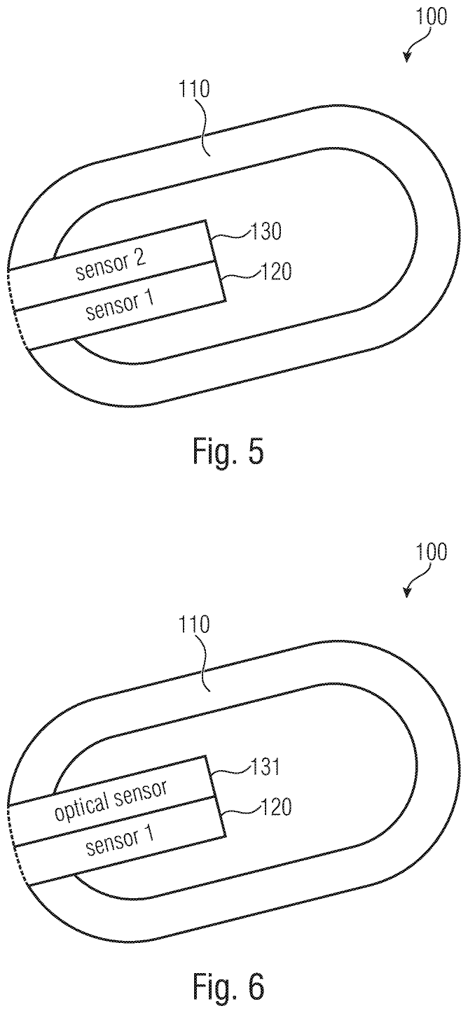

FIG. 5 shows such a sensor pill 100 in accordance with an embodiment comprising a sensor 120 and a second sensor 130.

In one embodiment, the two or more sensors may, for example, comprise an optical sensor 131.

FIG. 6 shows such a sensor pill 100 in accordance with an embodiment comprising an optical sensor 131 as a second sensor.

In accordance with an embodiment, the sensor pill 100 may additionally comprise a light source 140, for example, to support recording by the optical sensor 131.

Exemplarily, in one embodiment, the optical sensor 131 may be configured for recording visible light (for example visible for the human eye).

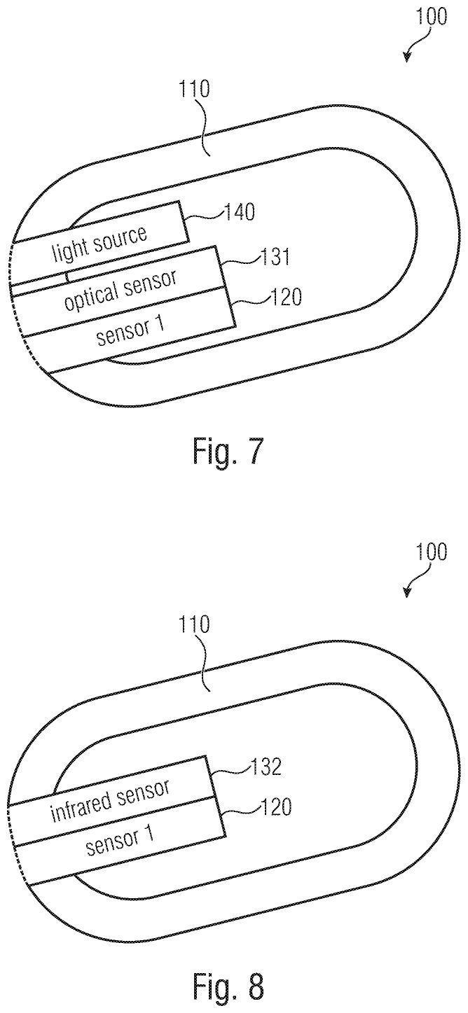

FIG. 7 shows a sensor pill 100 in accordance with an embodiment, comprising an optical sensor 131 as a second sensor, additionally comprising a light source 140.

The light source may, for example, be one or more LEDs.

In one embodiment, the optical sensor may, for example, be an infrared sensor 132.

FIG. 8 shows a sensor pill 100 in accordance with an embodiment, in which the optical sensor is an infrared sensor 132.

In accordance with an embodiment, the two or more sensors may, for example, comprise an ultrasonic sensor 133.

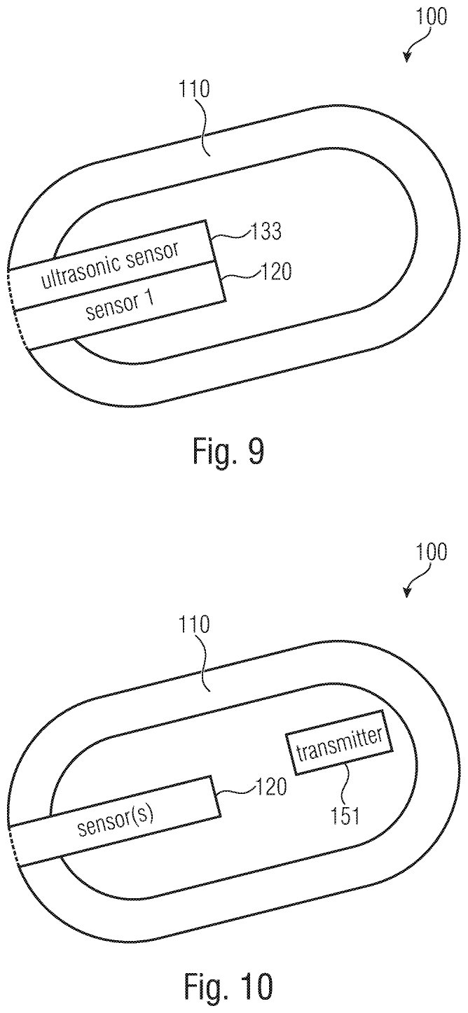

FIG. 9 shows a sensor pill 100 in accordance with an embodiment, in which the optical sensor is an ultrasonic sensor 133.

In one embodiment, the sensor pill 100 may additionally comprise a transmitter 151, for example, to transmit data to a receiver. Thus, the data may, for example, comprise a digital recording detected by at least one of the one or more sensors 120, 121, 122, 125, 130, 131, 132, 133.

FIG. 10 shows a sensor pill 100 comprising a transmitter 151 in accordance with an embodiment.

In accordance with an embodiment, the transmitter 151 may, for example, be configured to transmit data by means of near-field communication.

Data transmission by means of near-field communication offers particular advantages for advantageous embodiments:

In near-field communication, communication will only be possible if the receiver 251 is close to the transmitter 151. In one embodiment, the receiver 251 of near-field communication can be moved along on the body of the patient.

Data transmission will only be possible if the receiver 251 of near-field communication is close to the transmitter 151 of the near-field communication, in this case close to the transmitter 151 of the sensor pill 100. The examiner obtains information on the (approximate) position of the sensor pill 100 within the body of the patient:

If the data transmission is performed, the receiver 251 is close to the sensor pill 100. This also reveals where the sensor pill 100 is located when detecting the noises or the vibrations, for example.

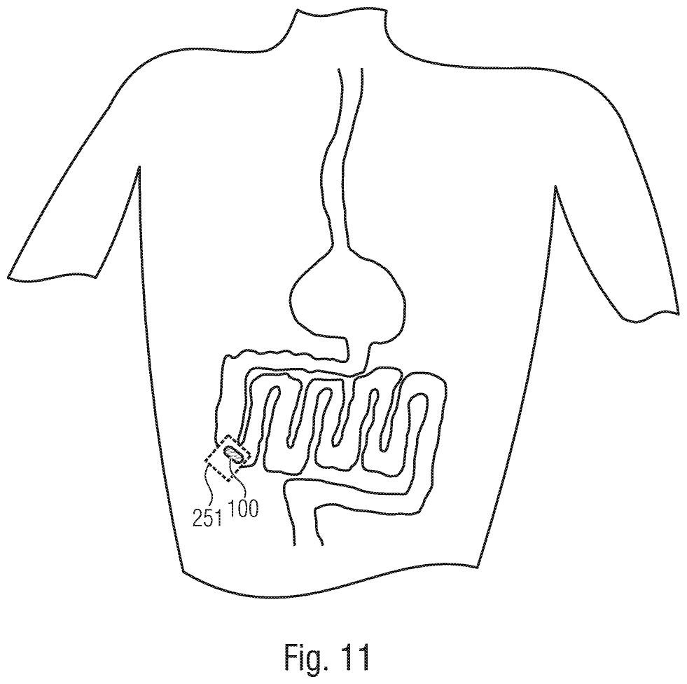

FIG. 11 exemplarily shows how a receiver 251 localizes the position of the sensor pill 100 by means of near-field communication: The data transmission will only take place if the receiver is close to the sensor pill 100.

Another advantage of a transmitter 151 transmitting data by means of near-field communication is that the power supply of the transmitter 151 can be done via receiver 251. This means that no separate power supply is required for the data transmission.

In another embodiment, the power supply of the one or more sensors 120 can be done via an external power supply of the transmitter 151 for transmitting data by means of near-field communication.

This means that, in one embodiment, it is possible for the sensor pill not to comprise a separate current source. Instead, the external near-field communication receiver 251 feeds current into the transmitter 151 of the near-field communication, which is how the one or more sensors 120 also receive a current.

Exemplarily, the one or more sensors 120 will only detect acoustic noises or vibrations if they are supplied with current via an external receiver of the near-field communication.

In another embodiment, the sensor pill 100 also comprises one or more capacitors suitable for being charged by means of an external receiver of the near-field communication so that detecting acoustic noises or vibrations also remains possible after removing the external receiver of the near-field communication.

In one embodiment, the data detected by the sensor pill 100 are transmitted directly when detected.

In another embodiment, the detected data are buffered in a storage (not shown) of the sensor pill 100 and transmitted to a receiver at a later point in time.

In one embodiment, the transmitter 151 may, for example, be configured for wireless communication with a local network.

In accordance with an embodiment, the casing 110 of the sensor pill 100 may, for example, comprise rounded edges. This avoids injuries for the patient when applying the sensor pill 100.

In one embodiment, the casing 110 of the sensor pill 100 may, for example, be configured not to comprise corners. Again, injuries for the patient when applying the sensor pill 100 are avoided.

In accordance with an embodiment, the sensor pill 100 may, for example, comprise a separate current source 160 for power supply.

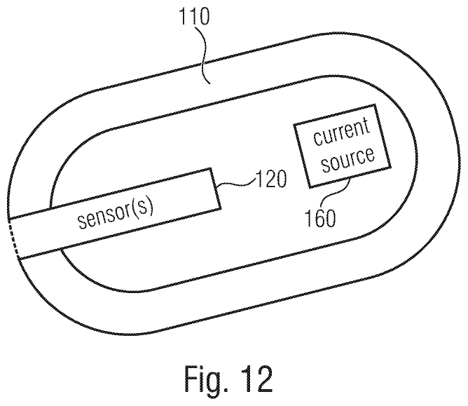

FIG. 12 shows a sensor pill 100 having a current source in accordance with an embodiment.

In one embodiment, the current source 160 may, for example, comprise a battery or an accumulator.

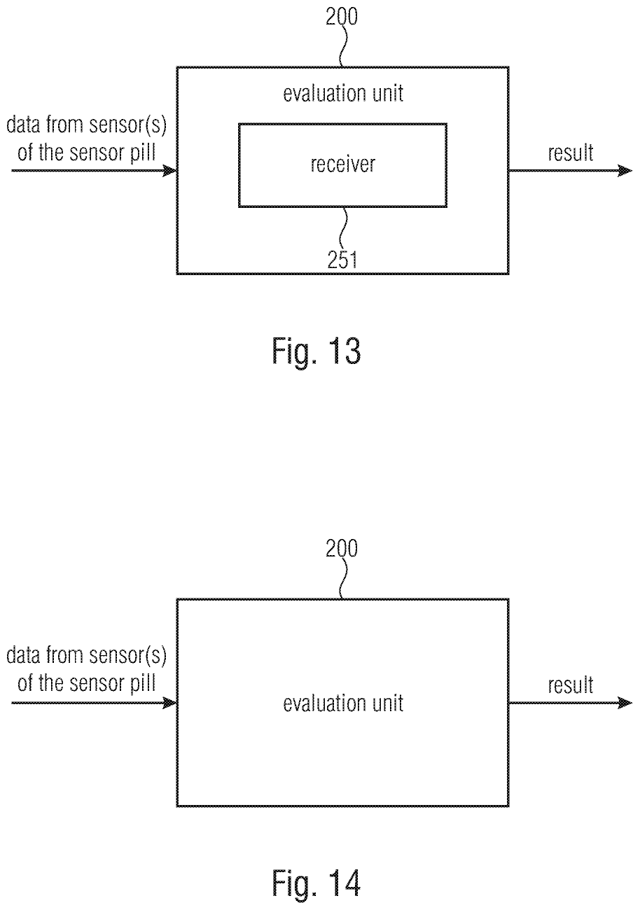

FIG. 13 shows an evaluation unit 200 in accordance with an embodiment.

The evaluation unit 200 is configured to evaluate data detected by at least one of the one or more sensors 120 of the sensor pill 100 described above.

In accordance with an embodiment, the evaluation unit 200 may, for example, be configured to evaluate the data such that the evaluation unit 200 provides a result indicating a hint as to which disease a patient who before has swallowed the sensor pill 100 described above may suffer from.

In one embodiment, the evaluation unit 200 may, for example, be configured for localization and/or detection and/or classification of one or more acoustic sources and/or one or more vibration sources.

In accordance with an embodiment, the evaluation unit 200 may, for example, be configured to use a machine learning method for localization and/or detection and/or classification of the one or more acoustic sources and/or the one or more vibration sources.

In one embodiment, the evaluation unit 200 may, for example, be configured to employ a neural network as the machine learning method.

In accordance with an embodiment, an evaluation unit 200 may, for example, additionally comprise a receiver 251 to receive data from the sensor pill 100 described above. Thus, the data may, for example, comprise a digital recording detected by at least one of the one or more sensors of the sensor pill 100 described above.

FIG. 14 shows an evaluation unit 200 in accordance with an embodiment which additionally comprises a receiver for receiving data from a transmitter 151 of the sensor pill 100.

In one embodiment, the receiver 251 may, for example, be configured for receiving data by means of near-field communication.

In accordance with an embodiment, the receiver 251 may, for example, be configured for wireless communication with a local network.

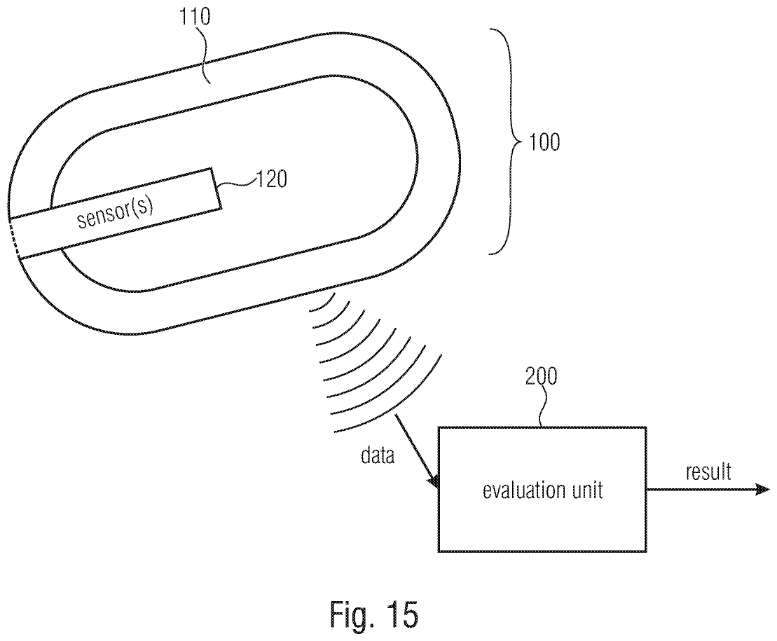

FIG. 15 shows a system in accordance with an embodiment.

The system comprises the sensor pill 100 in accordance with an embodiment described above.

Additionally, the system comprises the evaluation unit 200 in accordance with an embodiment described above.

The evaluation unit 200 is configured to evaluate data detected by the one or more sensors of the sensor pill 100.

Special embodiments will be described below.

In accordance with an embodiment, an acoustic sensor pill 100 is provided, which comprises a microphone, or an array of microphones and/or one or more acceleration sensors as a sensor.

In one embodiment, the acoustic sensor pill 100 is configured to record vibro-acoustic signals within the body and transmit the same to the outside.

In a special embodiment, the sensor pill 100 is configured to transmit the recorded data to a PC, for example.

In one embodiment, a patient takes in the inventive audio pill.

The pill moves through the body and can thus provide an extensive acoustic health image of many body regions.

The pill records acoustic signals from inside the body, for example.

Further embodiments provide algorithms for localizing, detecting, classifying and separating acoustic sources.

In embodiments, an algorithm for an acoustic sensor pill 100 is provided, which determines and/or monitors the health state of internal organs based on machine learning, for example, to ensure a symptom-free riskless examination for the patient, for example.

Exemplarily, the provided algorithms are based, for example, on machine learning to identify the origin, type and cause of the recorded audio signals and to allow drawing conclusions as to healthy or pathological alterations, characteristics, for example.

In embodiments, the provided algorithm comprises a neural network for machine learning, wherein one or more regions of the recorded signal are applied to the input of the neural network, and wherein the neural network outputs as an output a respective disease which the patient in which the one or more regions of the recorded signal were recorded suffers from.

In one embodiment, an algorithm in accordance with an embodiment determines a potential disease of the patient relating to a recorded audio signal.

One of the advantages is obtaining the most objective evaluation possible of acoustic signals of the body to be able to identify a disease.

Examples of application are in medical technology, for example in the field of intelligent non-invasive diagnostics.

Although some aspects have been described in the context of an apparatus, it is clear that these aspects also represent a description of the corresponding method such that a block or element of an apparatus is to be understood to be also a respective method step or a feature of a method step. Analogously, aspects described in the context of or as a method step also represent a description of a corresponding block or item or feature of a corresponding apparatus. Some or all of the method steps may be executed by (or using) a hardware apparatus, like, for example, a microprocessor, a programmable computer or an electronic circuit. In some embodiments, some or several of the most important method steps may be executed by such an apparatus.

Depending on certain implementation requirements, embodiments of the invention can be implemented in hardware or in software or at least partly in hardware or at least partly in software. The implementation can be performed using a digital storage medium, for example a floppy disk, a DVD, a Blu-Ray disc, a CD, an ROM, a PROM, an EPROM, an EEPROM or a FLASH memory, a hard drive or another magnetic or optical memory having electronically readable control signals stored thereon, which cooperate or are capable of cooperating with a programmable computer system such that the respective method is performed. Therefore, the digital storage medium may be computer-readable.

Some embodiments according to the invention include a data carrier comprising electronically readable control signals, which are capable of cooperating with a programmable computer system such that one of the methods described herein is performed.

Generally, embodiments of the present invention can be implemented as a computer program product with a program code, the program code being operative for performing one of the methods when the computer program product runs on a computer.

The program code may, for example, be stored on a machine-readable carrier.

Other embodiments comprise the computer program for performing one of the methods described herein, wherein the computer program is stored on a machine readable carrier. In other words, an embodiment of the inventive method is, therefore, a computer program comprising a program code for performing one of the methods described herein, when the computer program runs on a computer.

A further embodiment of the inventive methods is, therefore, a data carrier (or a digital storage medium or a computer-readable medium) comprising, recorded thereon, the computer program for performing one of the methods described herein. The data carrier or the digital storage medium or the computer-readable medium is typically tangible and/or non-volatile.

A further embodiment of the inventive method is, therefore, a data stream or a sequence of signals representing the computer program for performing one of the methods described herein. The data stream or the sequence of signals may, for example, be configured to be transferred via a data communication connection, for example via the Internet.

A further embodiment comprises processing means, for example a computer, or a programmable logic device, configured to or adapted to perform one of the methods described herein.

A further embodiment comprises a computer having installed thereon the computer program for performing one of the methods described herein.

A further embodiment according to the invention comprises an apparatus or a system configured to transfer a computer program for performing one of the methods described herein to a receiver. The transmission can be performed electronically or optically. The receiver may, for example, be a computer, a mobile device, a memory device or the like. The apparatus or system may, for example, comprise a file server for transferring the computer program to the receiver.

In some embodiments, a programmable logic device (for example a field-programmable gate array, FPGA) may be used to perform some or all of the functionalities of the methods described herein. In some embodiments, a field-programmable gate array may cooperate with a microprocessor in order to perform one of the methods described herein. Generally, in some embodiment the methods are performed by any hardware apparatus. This can be universally applicable hardware, such as a computer processor (CPU), or hardware specific for the method, such as ASIC.

While this invention has been described in terms of several embodiments, there are alterations, permutations, and equivalents which will be apparent to others skilled in the art and which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1. A sensor pill suitable for being swallowed by a patient, the sensor pill comprising:
a casing of the sensor pill, and
one or more sensors, wherein the one or more sensors comprise a microphone,
wherein said microphone is a microphone of a plurality of microphones forming an array of microphones,
the sensor pill comprising the array of microphones,
wherein the array of microphones is configured to determine first directional information relating to a first of the acoustic noises detected by the array of microphones, and
wherein the array of microphones is configured to determine second directional information relating to a second of the acoustic noises detected by the array of microphones.

2. The sensor pill in accordance with claim 1,
wherein the one or more sensors comprise an acceleration sensor.

3. The sensor pill in accordance with claim 2,
wherein the acceleration sensor is configured to detect vibrations originating from inside the body of the patient.

4. The sensor pill in accordance with claim 1,
wherein the microphone is configured to record acoustic noises originating from inside the body of the patient.

5. The sensor pill in accordance with claim 1,
wherein the one or more sensors are two or more sensors.

6. The sensor pill in accordance with claim 5,
wherein the two or more sensors comprise an optical sensor.

7. The sensor pill in accordance with claim 6,
the sensor pill additionally comprising a light source to support recording by the optical sensor.

8. The sensor pill in accordance with claim 6,
wherein the optical sensor is an infrared sensor.

9. The sensor pill in accordance with claim 5,
wherein the two or more sensors comprise an ultrasonic sensor.

10. The sensor pill in accordance with claim 1,
the sensor pill additionally comprising a transmitter to transmit data,
wherein the data comprise a digital recording detected by at least one of the one or more sensors.

11. The sensor pill in accordance with claim 10,
wherein the transmitter is configured for data transmission by means of near-field communication.

12. The sensor pill in accordance with claim 11,
the sensor pill being configured such that power supply of the one or more sensors takes place via an external power supply of the transmitter for data transmission by means of near-field communication.

13. The sensor pill in accordance with claim 1,
wherein the casing of the sensor pill comprises rounded edges.

14. The sensor pill in accordance with claim 1,
wherein the casing of the sensor pill does not comprise corners.

15. The sensor pill in accordance with claim 10,
wherein the transmitter is configured for wireless communication with a local network.

16. The sensor pill in accordance with claim 12,
the sensor pill not comprising a separate current source.

17. The sensor pill in accordance with claim 1,
the sensor pill comprising a current source for power supply.

18. The sensor pill in accordance with claim 17,
wherein the current source comprises a battery or an accumulator.

19. A system comprising:
a sensor pill suitable for being swallowed by a patient, the sensor pill comprising:
a casing of the sensor pill, and
one or more sensors, wherein the one or more sensors comprise a microphone,
wherein said microphone is a microphone of a plurality of microphones forming an array of microphones,
the sensor pill comprising the array of microphones,
wherein the array of microphones is configured to determine first directional information relating to a first of the acoustic noises detected by the array of microphones, and
wherein the array of microphones is configured to determine second directional information relating to a second of the acoustic noises detected by the array of microphones, and
an evaluation unit,
the evaluation unit being configured to evaluate data detected by at least one of the one or more sensors of the sensor pill.

20. The system in accordance with claim 19,
the evaluation unit being configured to evaluate the data such that the evaluation unit provides a result indicating a hint as to which disease a patient who has swallowed the sensor pill may suffer from.

21. The system in accordance with claim 19, the evaluation unit being configured for localization and/or for detection and/or for classification of one or more acoustic sources and/or one or more vibration sources.

22. The system in accordance with claim 21,
the evaluation unit being configured to use a machine learning method for localization and/or for detection and/or for the classification of the one or more acoustic sources and/or the one or more vibration sources.

23. The system in accordance with claim 22,
the evaluation unit being configured to employ a neural network as the machine learning method.

24. The system in accordance with claim 19,
the evaluation unit further comprising a receiver to receive data from the sensor pill,
wherein the data comprise a digital recording detected by at least one of the one or more sensors of the sensor pill.

25. The system in accordance with claim 24,
wherein the receiver is configured for receiving data by means of near-field communication.

26. The system in accordance with claim 24,
wherein the receiver is configured for wireless communication with a local network.

27. A method comprising:
swallowing a sensor pill by a patient, the sensor pill comprising a casing and one or more sensors which comprise a microphone, and
detecting an acoustic noise and/or a vibration by at least one of the one or more sensors of the sensor pill,
wherein said microphone is a microphone of a plurality of microphones forming an array of microphones,
the sensor pill comprising the array of microphones,
wherein the array of microphones is configured to determine first directional information relating to a first of the acoustic noises detected by the array of microphones, and
wherein the array of microphones is configured to determine second directional information relating to a second of the acoustic noises detected by the array of microphones.

28. The method in accordance with claim 27,
wherein the one or more sensors comprise an acceleration sensor which detects vibrations originating from inside the body of the patient.

29. The method in accordance with claim 27,
the method comprising evaluating the detected acoustic noises and/or vibrations by an evaluation unit.

30. A non-transitory digital storage medium having stored thereon a computer program for performing a method comprising:
swallowing a sensor pill by a patient, the sensor pill comprising a casing and one or more sensors which comprise a microphone, and
detecting an acoustic noise and/or a vibration by at least one of the one or more sensors of the sensor pill,
wherein said microphone is a microphone of a plurality of microphones forming an array of microphones,
the sensor pill comprising the array of microphones,
wherein the array of microphones is configured to determine first directional information relating to a first of the acoustic noises detected by the array of microphones, and
wherein the array of microphones is configured to determine second directional information relating to a second of the acoustic noises detected by the array of microphones,
when said computer program is run by a computer.
